(19) United States
(12) Patent Application Publication
(10) Pub. No.: US 2012/0150248 A1
(43) Pub. Date: Jun. 14, 2012

(54) Battery pack for an electronic device

(76) Inventors: Benny Sangyong Chi, Dumont, NJ (US); Gintaras A. Vaisnys, Chicago, IL (US)

(21) Appl. No.: 12/928,326

(22) Filed: Dec. 8, 2010

(51) Int. Cl. A61N 1/39

(52) U.S. Cl. 607/5

(57) ABSTRACT

A removable battery pack is disclosed for a portable medical device, such as an automated external defibrillator. The removable battery pack interacts with the portable medical device using an electrical connector that creates a watertight connection. The connection is created by a gasket that is not compressed in the direction of movement of the removable battery pack. As a result, the gasket does not increase the force necessary to engage the removable battery pack in the portable medical device.

### TECHNICAL FIELD

The present invention relates to electronic devices that use replaceable battery packs. More specifically, the invention relates to a waterproof battery pack that interfaces with the electronic device such that a watertight seal is created between the battery pack and the electronic device.

### BACKGROUND OF THE INVENTION

External defibrillators are emergency medical devices designed to supply a controlled electric shock (i.e., therapy) to a person's (e.g., victim's) heart during cardiac arrest. This electric shock is delivered via pads electrically connected with the external defibrillator and in contact with the person's body.

To provide a timelier rescue attempt for a person experiencing cardiac arrest, some external defibrillators have been made portable, by utilizing battery power (or other self-contained power supplies). In addition, many portable external defibrillators have programming to make medical decisions making possible operation by rescuers who are non-medical personnel.

These portable external defibrillators, commonly known as automated external defibrillators (AEDs), including automatic and semi-automatic variants, have gained acceptance by those outside the medical profession and have been deployed in myriad locations outside of traditional medical settings. Due to the life saving benefits of AEDs, more and more non-medical users are purchasing and deploying AEDs in their respective environments. This allows for a rescue attempt without the delay associated with bringing the person to a medical facility, or bringing a medical facility to the person (e.g., a life support ambulance).

AEDs use batteries for power. As those skilled in the art will appreciate, when an AED is used, a significant amount of energy is drained from the battery. But also, energy is drained from the batteries even when the AED is perceived to be off by a user. More precisely, AEDs perform a number of self-diagnostic tests to assure they will work when called upon, and, in addition, there is a natural loss of energy simply due to the passage of time. As a result, AEDs typically employ replaceable battery packs (i.e., a single unit containing multiple cells).

Replacement of batteries, or battery packs, must be user friendly and maintain the integrity of the AED. A significant issue in battery pack replacement is the force that must be exerted on the battery pack to secure it in the AED and/or the integrity of a seal that is needed to avoid infiltration of the battery pack or AED unit by undesirable substances, such as water or dust.

The present invention is an improved battery pack. Furthermore, other desirable features and characteristics of the present invention will become apparent for the subsequent detailed description of the invention and the appended claims, taken in conjunction with the accompanying drawings and this background of the invention.

### SUMMARY OF THE INVENTION

The invention is a removable battery pack for a portable medical device, such as an automated external defibrillator. The removable battery pack interacts with the portable medical device using an electrical connector that creates a watertight connection. The connection is created by a gasket that is not compressed in the direction of insertion of the removable battery pack. As a result, the gasket does not increase the force necessary to engage the removable battery pack in the portable medical device making the replacement of the battery pack more user friendly.

Other features, attainments, and advantages will become apparent to those skilled in the art upon a reading of the description when taken in conjunction with the accompanying drawings.

### BRIEF DESCRIPTION OF THE DRAWINGS

FIG. 1 is a front perspective drawing of an automated external defibrillator (AED).

FIG. 2 is a drawing of the back of the AED shown in FIG. 1.

FIG. 3 is an enlarged drawing of a connection point located in a battery slot on the AED, see circle 3 in FIG. 2.

FIG. 4A is a cross-sectional view taken along line 4A shown in FIG. 3.

FIG. 4B is a cross-sectional view taken along line 4B shown in FIG. 3.

FIG. 5 is an enlarged view of a gasket groove shown in FIGS. 4A and 4B, see circle 5 in FIG. 4B.

FIG. 6 is a front perspective view of a latch used to secure a battery pack in the battery slot.

FIG. 7 is a back perspective view of the latch shown in FIG. 6.

FIG. 8 is a perspective view of a battery pack showing the bottom.

FIG. 9 is an expanded view of the battery pack shown in FIG. 8 in the area of an electrical connector, see circle 9 in FIG. 8.

FIG. 10 is a cut-away view of the AED in the area of the battery slot proximate a latch, see FIG. 3 taken along line 10, showing a battery pack as it initially interacts with the latch when placed in the battery slot.

FIG. 11 is the same view as that shown in FIG. 10, but showing the back pack inserted deeper in the battery slot.

FIG. 12 is a cut-away view of a gasket positioned on the battery pack interacting with a bearing surface on the AED, see FIGS. 4A and 4B for further gasket groove details.

### DETAILED DESCRIPTION

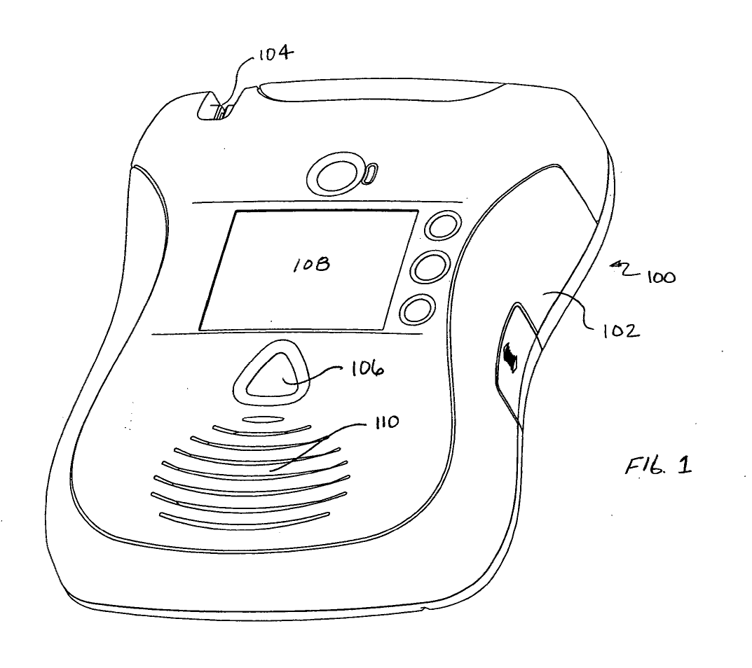

As shown in FIG. 1, a portable automated external defibrillator (AED), generally referred to by reference no. 100, includes a case 102 having defibrillation electronics and programming therein. Pads (not shown) connect to the AED 100 via a connector 104 (only one-side shown). Portable AEDs are well known in the art.

The depicted AED 100 is a semi-automatic AED, as a shock is sent to a victim via the pads by depressing a shock button 106. Also, this illustrative AED 100 has a video display 108 for displaying visual information, such as user instructions, and a speaker 110 for providing audio instructions.

Continuing with FIG. 2, as shown on the back of the AED 100, a slot 112 is provided for accepting a removable battery pack, discussed below. The slot 112 includes a first part 120A of the electrical connector 120 (the electrical connector as a whole is referred to by reference no. 120 with elements, in this case parts, that make up the electrical connector being referred to by reference no. 120 with an appropriate suffix, such as A and B), a spring 114, and a latch 118.

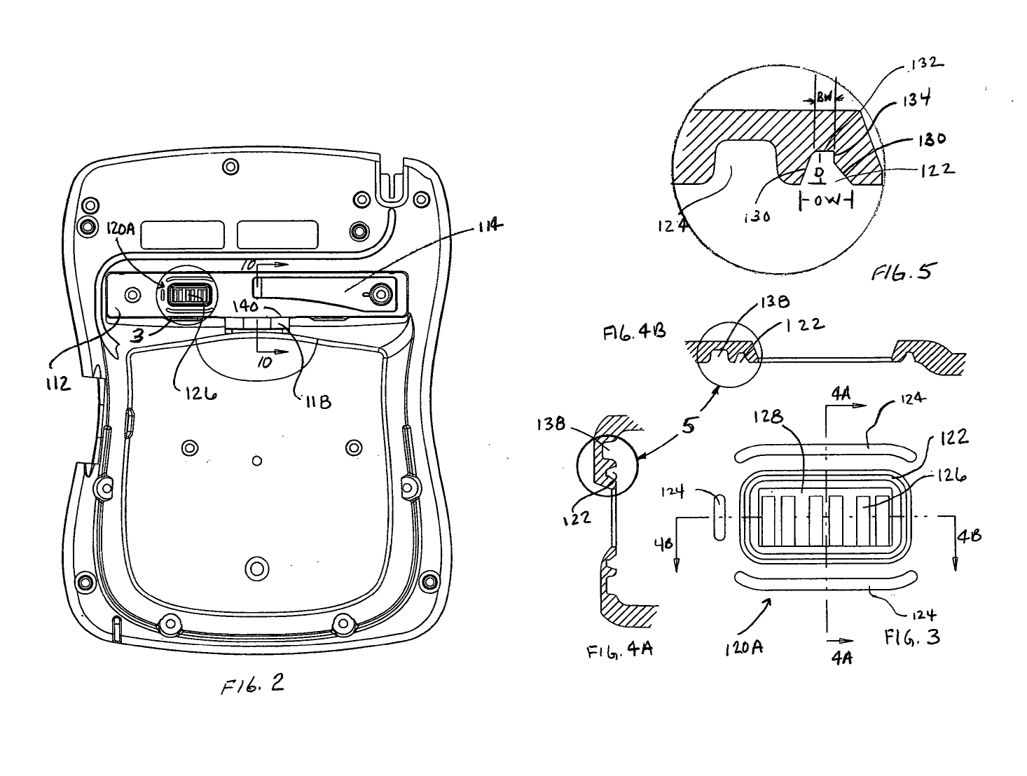

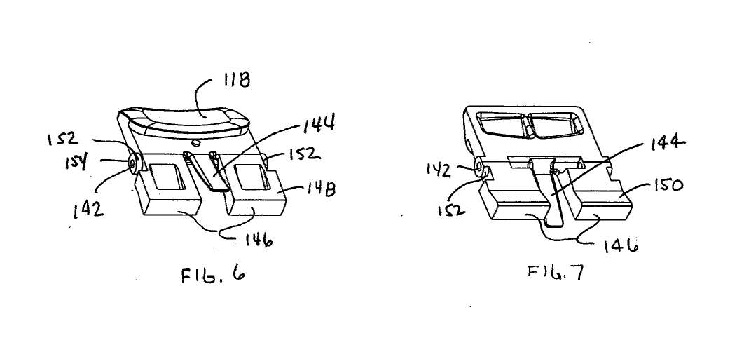

The first part 120A of the electrical connector 120, which is shown in more detail in FIG. 3, as illustrated, includes a gasket groove 122, depressions 124, and spring contacts 126. More specifically, the gasket groove 122 surrounds an opening 128 in the AED 100 wherein the spring contacts 126, for connecting the battery to the defibrillation electronics, are positioned.

Continuing with FIGS. 4A, B and 5, the gasket groove 122 has a cross-section having a depth D, an opening width OW and a bottom width BW, which is less that the opening width. The gasket groove 122 has sides 130 and a bottom 132. As a result of the opening width OW being less than the bottom width BW, the gasket groove 122 cross-section is tapered. However, the taper ends above the bottom 132 thereby defining a bearing surface 134. As illustrated, the bearing surface 134 is generally perpendicular to the bottom surface 132 and located on a side 130 of the gasket groove 122 that is proximate the opening 128.

The depressions 124 are positioned around the gasket groove 122 on the side away from the opening 128. In this illustrative example, there are three depressions.

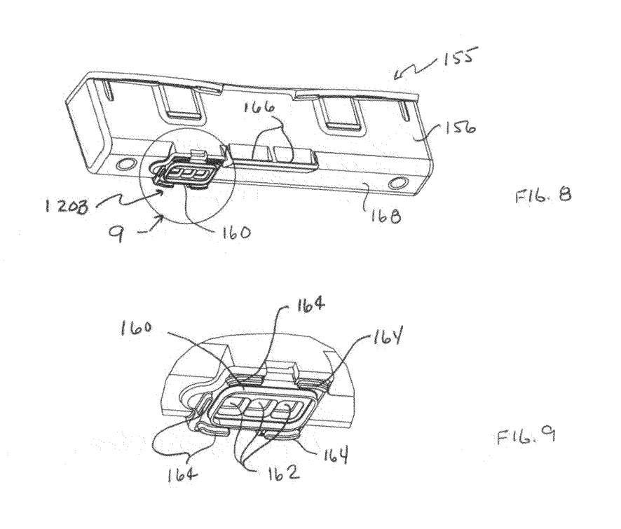

As shown in FIGS. 6 and 7, the latch 118, which is located on a side 140 of the slot 112 (See FIG. 2), is designed to interact with the battery pack, discussed below, to secure the battery pack within the slot 112. The latch 118 includes a pivot 142, a spring 144, a latching surface 146, a first surface 148, and a second surface 150.

The pivot 142 is the point about which the latch 118 is secured to the AED. The pivoting of the latch 118 about the pivot 142 permits the latching surface 146 of the latch 118 to engage and disengage the latching surface, which interacts with the battery pack to secure it in the slot 112. The spring 144 provides a bias to the latch 118, making it more user friendly. More specifically, the spring 144 makes the latch 118 self-locking, which is discussed below.

The pivot 142 has a round bearing surface 152 and holes 154. Typically in this design, when the latch 118 is pivoted it rides on the bearing surfaces 152. The holes 154 provide a passage for an axle (single or multi-part), which would positively secure the latch 118 to the AED 100. Each bearing surface 152 could be snapped into cooperating openings (not shown) in the AED 100. It should be appreciated that either or both of the above structures could be used.

Continuing with FIG. 8, a battery pack, generally referred to by reference number 155, includes a container 156 for holding a number of cells (not shown), a second part 120B of the electrical connector 120 of the electrical connector 120, and abutment surfaces 166.

As shown in FIG. 8 and in greater detail in FIG. 9, the second part 120B of the electrical connector 120 includes a gasket 160 and a second set of contacts 162. The second set of contacts 162, in this case fixed surfaces, cooperate with the spring contacts 126 to connect the battery pack 155 to the AED. Both the second set of contacts 162 and the spring contacts 126 conduct electricity.

The gasket 160, which is a rubber, such as urethane, can be surface mounted or in a groove. Additional characteristics of the gasket 160 are discussed below.

The second part 120B of the electrical connector 120 further includes a guard 164, which projects outwardly from the container 156. The illustrated guard 164, which has multiple posts, has a height sufficient to protect the gasket 160, which also projects outwardly from the container 156. More precisely, the height of the guard 164 is such that the guard can rest on a surface, such as a table, and the gasket 160 will not touch the surface. In essence, the guard 164 protects the gasket 160 from being damaged when the battery pack 155 is not within the slot 112.

The abutment surface 166 on the battery pack 155 cooperates with the abutment surfaces 146 on the latch 118 to secure the battery pack in the AED.

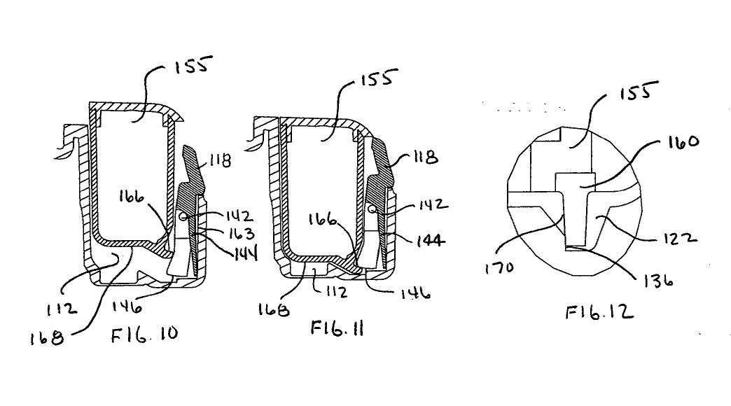

As shown in FIG. 10, the abutment surface 146 on the latch 118 projects outwardly from a wall 168 of the slot 112 as a result of the spring 144. Upon insertion of the battery pack 155 into the slot 112, the battery pack travels down into the slot and impacts the latch 118. As the latch 118 is impacted, it rotates about its pivot 142 so the battery pack 155 can continue downward in the slot 112.

At some point, a surface 168 (see FIG. 10) of the battery pack 155, in this case the bottom surface, will impact the spring 114 (see FIG. 2). Additionally, at some point the contacts 126 (see FIG. 8) of the electrical connector 120 will impact the spring contacts 126 (see FIG. 2). To allow for a generally parallel descent of the battery pack 155 into the slot 112, the spring 114 and the spring contacts 126 should be engaged approximately simultaneously. It should also be appreciated that the spring 114 and spring contacts 162 are separated by the abutment surface 166 (see FIG. 8) which further supports creating a generally parallel descent.

Continuing with FIG. 11, the abutment surfaces 166 on the battery pack 155 will pass the abutment surfaces 146 on the latch 118. As the abutment surfaces pass, the spring 144 on the latch 118 will force the abutment surfaces 146 on the latch 118 to engage the cooperating abutment surfaces 166 on the battery pack 155. This engagement secures the battery pack 155 in the slot 112.

Continuing with FIG. 12, which shows the gasket 160 in its final position on the bearing surface 136, as the descent of the battery pack 155 continues but prior to the engagement of the abutment surfaces, the gasket will engage the bearing surface. To assure the best fit possible of the gasket 160 against the bearing surface 136, as an edge 170 of the gasket initially contacts the bearing surface, the gasket should distend laterally outward (i.e., away from the bearing surface). As the edge 170 of the gasket 160 continues down the bearing surface 136, the characteristics of the material from which the gasket is made should allow the gasket to conform to the bearing surface. Thus, there could be a slight lateral compression of the gasket 160 (i.e., the thickness of the gasket in the area where it is in contact with the bearing surface may be slightly less than the area where it is not).

As shown in FIG. 12, when the gasket 160 conforms to the bearing surface 136 it creates a watertight seal along the bearing surface. As a result, water cannot enter the AED 100 through the electrical connection 120 when a battery pack 155 is installed. In addition, the placement of the gasket 160 between the bearing surface 136 and any water seeking entry into the AED 100 means that water pressure with further increase the integrity of the seal.

Further, as shown in FIG. 12, to avoid compressing the gasket 160 (i.e., reducing its height), which would added resistance to inserting the battery pack 155, the height of the gasket 160 should be less than the depth of the gasket groove 122. Thus, the gasket 160 should not bottom out in the gasket groove 122. It should be appreciated that the extra height guard 164, relative to the gasket 160, will interfere with the downward movement of the battery pack 155. As a result, depressions 124 are provided to accept the guard 164. There is no requirement the guard 164 contact any of the surfaces on the depressions. The depressions are provided for clearance.

Referring to FIG. 11, as those skilled in the art will appreciate, after the abutment surfaces of the latch and battery pack engage, the battery pack 155 is held in the slot 122 but there is significant energy in the spring 114 and spring contacts 162, which if released will cause the battery pack 155 to move upward in the slot 112. In order to remove the battery pack 155 from the slot 112, a person pushes the latch 118 causing the latch to rotate about the pivot 142 that in turn disengages the abutment surfaces.

Although certain embodiments of the invention have been illustrated and described in considerable detail, it will be understood that this was only one example and the numerous changes in the details of the construction and arrangement may be made without departing from the spirit and scope of the invention.

What is claimed is:

1. A battery pack for a medical device comprising:
a container for holding a cell, the container defining an opening,
a gasket having a first height relative to the container and surrounding the opening;
a guard having a second height relative to the container and surrounding the opening;
wherein the second height is greater than the first height.

2. The battery pack of claim 1 further including a contact connected to the cell and positioned in the opening below the first height.

3. A medical device employing a removable battery pack comprising:
a medical device having
a case defining a battery pack slot, the slot having therein a first part of an electrical connector;
wherein the first part of an electrical connector includes spring contacts, surrounded by a gasket groove, the gasket groove having sides and a bottom
wherein a side has a bearing surface;
a battery pack dimensioned to slide into the slot, the battery pack having
a second part of the electrical connector to engage the first part of the electrical connector,
wherein the second part of the electrical connector has a gasket and contacts,
wherein the gasket is dimensioned to engage the bearing surface.

4. The medical device of claim 3 wherein the gasket groove has a tapering cross-section into the bearing surface.

5. The medical device of claim 3 wherein the gasket does not contact the bottom.
